(19) United States
(12) Patent Application Publication
(10) Pub. No.: US 2018/0289292 A1
(43) Pub. Date: Oct. 11, 2018

(54) Detection Systems Using Fingerprint Images for Type 1 Diabetes Mellitus and Type 2 Diabettes Mellitus

(71) Applicant: Ohio University, Athens, OH (US)

(72) Inventors: Molly R. Morris, Athens, OH (US); Bjoern C. Ludwar, Athens, OH (US)

(73) Assignee: Ohio University, Athens, OH (US)

(21) Appl. No.: 15/765,399

(22) PCT Filed: Sep. 30, 2016
(86) PCT No.: PCT/US2016/054636

§ 371 Date: Apr. 2, 2018

Related U.S. Application Data
(60) Provisional application No. 62/236,742, filed on Oct. 2, 2015.

(51) Int. Cl. A61B 5/1172; A61B 5/00; A61B 5/145

(52) U.S. Cl. CPC A61B 5/1172; A61B 5/1464; A61B 5/14532; A61B 5/0077

(57) ABSTRACT

Methods and kits for determining a propensity to develop Type 1 diabetes mellitus (T1DM) and for Type 2 diabetes mellitus (T2DM) in an individual by measuring an asymmetry of at least one captured fingerprint from the individual are described.

### CROSS-REFERENCE TO RELATED APPLICATIONS

This application claims the claims priority to U.S. provisional application Ser. No. 62/236,742 filed Oct. 2, 2015, the entire disclosure of which is expressly incorporated herein by reference.

### STATEMENT REGARDING FEDERALLY FUNDED SPONSORED RESEARCH

This invention not was made with any government support and the government has no rights in the invention.

### BACKGROUND OF THE INVENTION

Type 2 diabetes mellitus (T2DM) is the most common form of diabetes, affecting nearly 26 million US adults aged 20 years or older, with 1.5 million more adult cases diagnosed each year, as noted by Centers for Disease Control and Prevention in 2011. The CDC predicts that 1 in 3 Americans born in 2000 will develop diabetes in their lifetime. While millions of Americans have been diagnosed with T2DM, a serious problem is that many more are unaware they are at high risk. One-third to one-half of people with T2DM are left undiagnosed and, hence, untreated. It has been shown that aggressive lifestyle intervention can delay or prevent T2DM in those at high risk. It is also believed that earlier diagnosis and treatment can prevent or delay the serious complications related to the disease and improve health outcomes. Since one third of people have a complication from T2DM at the time they are diagnosed and duration of hyperglycemia is directly related to complications, the earlier diagnosis and intervention could have a significant impact on complication prevention.

With an 11% per year progression from pre-diabetes to T2DM there is clear a lag from conversion to diagnosis. However, the benefits of implementing preventive measures requires identifying those at risk before they develop T2DM, as 30-50% of individuals with newly diagnosed T2DM have already developed complications at the time of diagnosis.

T2DM is largely preventable and wholly treatable. A healthy lifestyle is very effective at preventing T2DM. Implementing preventive measures will be more successful once those at risk are identified. The currently available models for determining a risk of developing T2DM are based on several different factors, including being overweight, particularly if your body stores fat primarily in your abdomen, body mass index (BMI), waist circumference, history of high glucose levels, inactivity, family history, race, age (although T2DM is increasing dramatically among children, adolescents and younger adults), pre-diabetes, and gestational diabetes. However, all of these models are sometimes inadequate to identify everyone at risk.

In addition, T2DM is a disease with multiple genetic and environmental influences, making it difficult to determine any given individual's risk. If an individual knows they are at risk for T2DM, they can take necessary preventive measures to avoid or delay developing this disease and further prevent the complications of this disease.

Since T2DM is a disease with multiple genetic and environmental influences, such that even DNA sequencing of an individual's entire genome may not provide as predictive power as an indicator of an individual's growth strategy. Therefore, there is a great need for an inexpensive but comprehensive screening detection system for diabetes in humans.

In contrast, Type 1 diabetes mellitus (T1DM) is a form of diabetes mellitus that results from the autoimmune destruction of the insulin-producing beta cells in the pancreas. The subsequent lack of insulin leads to increased blood and urine glucose. The classical symptoms are polyuria, polydipsia, polyphagia and weight loss.

Even though T1DM is a considerably different disease than T2DM, like T2DM, T1DM is influenced by multiple environmental and genetic factors, including a relationship between environmental exposures in utero and risk of developing T1DM.

In spite of considerable research into therapies to diagnose and treat these T1DM and/or T2DM diseases, it remains difficult to treat effectively, and the mortality observed in patients indicates that improvements are needed in the diagnosis, treatment and prevention of this disease.

There is no admission that the background art disclosed in this section legally constitutes prior art.

### SUMMARY OF THE INVENTION

In a first broad aspect, there is described herein a detection system to determine a propensity to develop either Type 1 diabetes mellitus (T1DM) or Type 2 diabetes mellitus (T2DM) using the incidence of fluctuating asymmetry (FA) between homologous in fingerprints (e.g., the first finger on the right hand has a different wavelet analysis score than the first finger on the left hand). The degree of asymmetry is significantly greater in individuals with T1DM or T2DM.

Various objects and advantages of this invention will become apparent to those skilled in the art from the following detailed description of the preferred embodiment, when read in light of the accompanying drawings.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the U.S. Patent and Trademark Office upon request and payment of the necessary fees.

FIG. 1A: Two loop fingerprints (L1 and L2) and one whorl patterned fingerprint (R1) with vectors (black lines shown) for counting ridge counts (rc) using pattern analyses.

FIG. 1B: For the wavelet analyses, image of the prints are first cropped around the core point.

FIG. 1C: Each cropped image is then divided into four quadrants, and three levels of Haar wavelet decomposition are computed for each quadrant to obtain the transformed image shown.

FIG. 1D: Differences between prints (measure of symmetry) using both methods are shown. Pattern analysis ridge count difference (Δrc), and wavelet based methods Euclidian distance |×| of the feature vectors. The feature vectors are generated from the transformed image and consist of the standard deviations for each of the 4×3×3=36 high frequency decompositions. This feature vector is considered an overall description of the individual fingerprint. Note that the traditional ridge count assigns very high numbers to whorl patterns. (R1=27.5) despite the ridges actually being not very dense. The wavelet-based method better reflects the density of a print; R1 is more similar to L2 (somewhat less dense, |x|=317.7) than L (very dense, |x|=343.7).

FIG. 2 shows Table 1—Multinomial regression analyses for diabetes state by finger pair: Table 1A) T2DM as compared to controls, using ridge counts to assess asymmetry; Table 1B) T2DM as compared to controls, using wavelet analysis to assess asymmetry; and, Table 1C) T1DM as compared to controls, using wavelet analysis to assess asymmetry.

FIG. 3 shows Table 2—Comparisons of AUC scores for each finger in the subset of individuals over 40: Table 2A) Asymmetry measured with ridge counts; predicting T2DM; Table 2B) Asymmetry measured with wavelet analysis, predicting T2DM; and, Table 2C) Asymmetry measured with wavelet analysis, predicting T1DM.

FIG. 4 shows Table 3—Mean wavelet asymmetry scores by finger for individuals with T1DM as compared to T2DM.

FIGS. 5A-5C show ROC curves showing the performance of the fingerprint IV wavelet asymmetry scores in predicting (FIG. 5A) T2DM and (FIG. 5B) T1DM, and fingerprint V in predicting (FIG. 5C) T1DM.

FIG. 6 shows Table 4—Mean ridge count asymmetry scores for individuals with T1DM as compared to T2DM.

### DETAILED DESCRIPTION OF THE PREFERRED EMBODIMENT(S)

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

### Definitions

In order to facilitate review of the various embodiments of the disclosure, the following explanations of specific terms are provided:

Dermatoglyphics: The scientific study of fingerprints.

Directional Asymmetry: When a bilateral trait deviates from symmetry, and is more often larger on one side as compared to the other.

Fluctuating Asymmetry: The deviation from perfect bilateral symmetry that is directionally random.

Genome: The entirety of an organism's hereditary information.

Gestational diabetes: A condition in which the glucose regulation and resulting levels become abnormal while a woman is pregnant (assumption of normality prior to pregnancy).

Gestational stages: Timing of the development of a fetus during pregnancy.

Homologous finger:/fingerprint: The matching finger on the other hand (e.g., ring finger on right hand is homologous to the ring finger on left hand).

Leptokurtotic: A distribution with positive excess kurtosis, or with the tails of the distribution being very large.

Mesenchyme: A type of undifferentiated loose connective tissue.

Prediabetes: A condition of abnormal glucose regulations (impaired fasting glucose or impaired glucose tolerance) that is above normal but not diagnostic of diabetes.

Ridge Pattern: Friction ridge patterns are commonly described as one of three patterns: arch (˜5% of fingers), whorl (˜30-35% of fingers) or loop (˜60-65% of fingers).

Rolled Fingerprint: Impression of a single fingerprint taken by rolling the finger from one side of the nail to the opposing side of the nail.

Slap Fingerprint: Flat impression of the central part of fingerprints taken by typically pressing four fingers against a scanner or fingerprint card. Thumbs are typically printed separately.

Standard Deviation: The square root of the variance of a measure, and is used to express the variability of a population.

Type 1 diabetes (TIDM): Formerly termed Insulin-Dependent DM (IDDM) or juvenile diabetes). An autoimmune condition in which the insulin producing beta cells of the pancreas are destroyed, resulting in an individual not secreting sufficient insulin and therefore needing exogenous insulin to live.

Type 2 diabetes (T2DM): Formerly termed Non-Insulin-Dependent DM (NIDDM) or adult-onset diabetes). A condition that results from genetic abnormalities combined with environmental and lifestyle risks that results in abnormal glucose values that result from insulin resistance, abnormal glucose production from the liver, or impaired insulin secretion.

Volar Pads: Transient swellings of the mesenchyme under the epidermis on the palmar surface of the hands and soles of the feet of the human fetus. The size, height and shape are thought to determine the friction ridge pattern type and count of fingerprints.

Therapeutic: A generic term that includes both diagnosis and treatment. It will be appreciated that in these methods the “therapy” may be any therapy for treating a disease including, but not limited to, pharmaceutical compositions, gene therapy and biologic therapy such as the administering of antibodies and chemokines. Thus, the methods described herein may be used to evaluate a patient before, during and after therapy, for example, to evaluate the reduction in disease state.

Adjunctive therapy: A treatment used in combination with a primary treatment to improve the effects of the primary treatment.

Clinical outcome: Refers to the health status of a patient following treatment for a disease or disorder or in the absence of treatment. Clinical outcomes include, but are not limited to, an increase in the length of time until death, a decrease in the length of time until death, an increase in the chance of survival, an increase in the risk of death, survival, disease-free survival, chronic disease, metastasis, advanced or aggressive disease, disease recurrence, death, and favorable or poor response to therapy.

Decrease in survival: As used herein, “decrease in survival” refers to a decrease in the length of time before death of a patient, or an increase in the risk of death for the patient.

Patient: As used herein, the term “patient” includes human and non-human animals. The preferred patient for treatment is a human. “Patient,” “individual” and “subject” are used interchangeably herein.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Poor prognosis: Generally refers to a decrease in survival, or in other words, an increase in risk of death or a decrease in the time until death. Poor prognosis can also refer to an increase in severity of the disease, such as an increase in spread (metastasis) of the cancer to other tissues and/or organs.

Screening: As used herein, “screening” refers to the process used to evaluate and identify candidate agents that affect such disease.

General Description

Described herein is a useful screening or detection system that identifies individuals predisposed to develop diabetes mellitus (DM) (i.e., T1DM or T2DM).

In a first broad aspect, there is provided herein a method for predicting a likelihood of developing DM in an individual by:

detecting at least one asymmetry in homologous fingerprint images taken from an individual;

assigning a risk score to the asymmetry detected; and

predicting the likelihood of developing DM when the asymmetry score is assigned a high risk score; and/or

predicting a less likely chance of developing DM when the asymmetry score is assigned a low risk score.

In certain embodiments, the test expression level is determined by wavelet analysis of specific features from the fingerprint images.

In certain embodiments, the method further comprises designing a treatment plan based on the diagnosis. In certain embodiments, the method further comprises administration of a treatment based on the diagnosis. In certain embodiments, the method further comprises determining prognosis based on the diagnosis.

In one embodiment, there is described herein a system method for determining a propensity to develop diabetes mellitus (DM) in an individual. The system includes:

a) measuring an asymmetry between captured fingerprint images from homologous fingers of the individual by using wavelet analysis to determine a degree of a fluctuating asymmetry;

b) determining that an elevated amount of asymmetry measured in step (a) relative to the amount of asymmetry in a control sample shows the propensity to develop DM by setting a boundary value between a degree of asymmetry in homologous fingerprint images collected from a control population and a degree of asymmetry in the homologous fingerprint image collected from the individual as an evaluation criterion, and

c) determining the risk for developing DM being relatively high in a case where the degree of asymmetry of the homologous fingerprint image measured is high as compared to a control.

In another embodiment, there is described herein a method for determining whether an individual has a pre-disposition for developing DM, wherein the method comprises the steps of:

determining the presence or absence of asymmetry in homologous fingerprint images taken from the individual, and based on the presence or absence of such asymmetry; and,

determining whether the individual has a pre-disposition for developing DM, and, optionally, recommending a particular treatment for DM or pre-DM condition.

In another embodiment, there is described herein a method of determining whether an individual is at risk for developing, diabetes mellitus (DM), comprising:

receiving at least one set of homologous fingerprint images extracted from the individual;

measuring by wavelet analysis a level of asymmetry between the set of homologous fingerprint images;

comparing the level of asymmetry between the set of homologous fingerprint images of the individual to a control level of symmetry in normal fingerprint images; and

diagnosing whether the individual is at risk for developing, DM if the level of asymmetry between the homologous fingerprint images in the set from the individual is greater than the level of asymmetry in the corresponding control.

In certain embodiments, the asymmetry is measured any time after the birth of the individual. Also, in certain embodiments, the method further includes determining a point in time during gestation that the individual is most susceptible to environmental stressors that interact with the genes for diabetes. The method can also further include indicating when during gestation a therapeutic intervention aimed at decreasing the incidence of diabetes is beneficial. For example, the environmental stressor can be a mother's diabetes.

In certain embodiments, a first homologous fingerprint image is compared with a second homologous fingerprint image by calculating Euclidean or Manhattan distances between the first and second homologous fingerprint images. It is to be understood that other methods to determine the distance between two vectors are also within the contemplated scope of the present invention.

In another embodiment, there is described herein a method for determining whether or not an individual has an increased risk of developing diabetes mellitus (DM), comprising:

obtaining least one set of homologous fingerprint images from the individual;

conducting laboratory analysis of the sample so as to obtain symmetry data of the homologous fingerprint images, wherein the laboratory analysis is wavelet analysis; and

determining that the individual has increased risk of developing DM if the asymmetry data indicate that the set of homologous fingerprint images are more asymmetrical than a control; or determining that the individual has no increased risk of developing DM if the asymmetry data indicate that the set of homologous fingerprint images are not more asymmetrical than the control.

In certain embodiments, the method further includes the step of correlating the data with similar data from a reference population.

Also described herein is a medium for holding instructions for performing a method for determining whether an individual has a pre-disposition for developing DM.

Also described herein is an electronic system for use in determining whether an individual has a pre-disposition for developing DM.

In certain embodiments, the medium and/or electronic system is configured for receiving information associated with the individual and/or acquiring from a network such information associated with the individual.

Also described herein is a kit useful or determining the whether an individual is at risk for developing diabetes mellitus (DM). the kit can include a device for obtaining at least one image of at least one fingerprint of at least one finger of the individual, and for comparing the at least one obtained fingerprint to a control sample set using wavelet analysis; and instructions for the use of the fingerprint image in determining the risk of developing DM, wherein the instructions comprise providing directions to compare the wavelet analysis of the fingerprint image to a control.

In certain embodiments, the fingerprint images are laid down in a database, such as an internet database, a centralized or a decentralized database.

Also described herein is a system for evaluating a patient at risk of developing diabetes mellitus, the system comprising: a data storage device storing instructions for evaluating a patient at risk of developing diabetes mellitus; and a processor configured to execute the instructions to perform a method including: receiving patient-specific data regarding images of the patient's fingerprints; creating a stored image representing at least a portion of the fingerprint based on the received patient-specific data; and classifying the risk of developing diabetes mellitus based on the asymmetry in the patient's fingerprint images by conducting a wavelet analysis of characteristic within the patient's fingerprints; and, optionally, generating a treatment recommendation or risk assessment based on the wavelet analysis value of the fingerprint's asymmetry and the classification of the diabetes mellitus.

Also described herein is a computer-implemented method of determining a risk of developing diabetes mellitus in an individual, the method comprising: receiving patient-specific data regarding images of the patient's fingerprints; creating a stored image representing at least a portion of the fingerprint based on the received patient-specific data; and classifying the risk of developing diabetes mellitus based on the asymmetry in the patient's fingerprint images by conducting a wavelet analysis of characteristics within the patient's fingerprints; and, optionally, generating a treatment recommendation or risk assessment based on the wavelet analysis value of the fingerprint's asymmetry and the classification of the diabetes mellitus.

Also described herein is a non-transitory computer readable medium for use on a computer system containing computer-executable programming instructions for performing a method of determining a risk of developing diabetes mellitus in an individual, the method comprising: receiving patient-specific data regarding images of the patient's fingerprints; creating a stored image representing at least a portion of the fingerprint based on the received patient-specific data; and; classifying the risk of developing diabetes mellitus based on the asymmetry in the patient's fingerprint images by conducting a wavelet analysis of characteristic within the patient's fingerprints; and, optionally, generating a treatment recommendation or risk assessment based on the wavelet analysis value of the fingerprint's asymmetry and the classification of the diabetes mellitus.

Also described herein is an electronic system for use in determining whether an individual has a pre-disposition for developing DM, comprising: an image capturing device for determining the presence or absence of asymmetry in homologous fingerprints, and based on the presence or absence of such asymmetry, and a computer implemented system determining whether the individual has a pre-disposition for developing DM, and, optionally, recommending a particular treatment for DM or pre-DM condition.

In certain embodiments, the classifying comprises: measuring an asymmetry between one or more captured fingerprint images from homologous fingers of an individual, wherein the measuring comprises using wavelet analysis to determine a degree of a fluctuating asymmetry; and, calculating an amount of asymmetry relative to the amount of asymmetry in a control sample, wherein the calculating comprises setting a boundary value between: i) a degree of asymmetry in homologous fingerprint images collected from a control population; and ii) a degree of asymmetry in the homologous fingerprint image collected from the individual.

In certain embodiments, the asymmetry is measured any time after birth of the individual.

In certain embodiments, the first homologous fingerprint image is compared with the second homologous fingerprint image by calculating the Euclidean or Manhattan distances between the first homologous fingerprint image and the second homologous fingerprint image.

In certain embodiments, the fingerprints are of the fourth finger.

In certain embodiments, the homologous fingerprints are of the fourth and fifth fingers, and the DM is Type 2 diabetes mellitus.

In certain embodiments, the homologous fingerprints are of the third, fourth and/or fifth fingers, and the DM is Type 1 diabetes mellitus.

In particular aspects, described herein is a detection system where fluctuating asymmetry (FA) in homologous fingerprints is used to identify individuals with the propensity to develop DM.

The system herein provides advantages over methods that relied solely on ridge counts to detect differences in symmetry. Furthermore, different ridge patterns and the limited variation in ridge counts (typical range: 2 to 20) reduce the potential sensitivity of the analysis and increase the impact of an error during the somewhat subjective counting procedure. In contrast, the system herein uses wavelet based analysis method, either alone, or in parallel with traditional ridge counts, in order to avoid these limitations.

Additionally, the comparison of the asymmetry across different homologous fingers provides valuable information regarding the timing of gestational environmental influences, which also leads to a better indication of when screening for gestational diabetes would be most valuable.

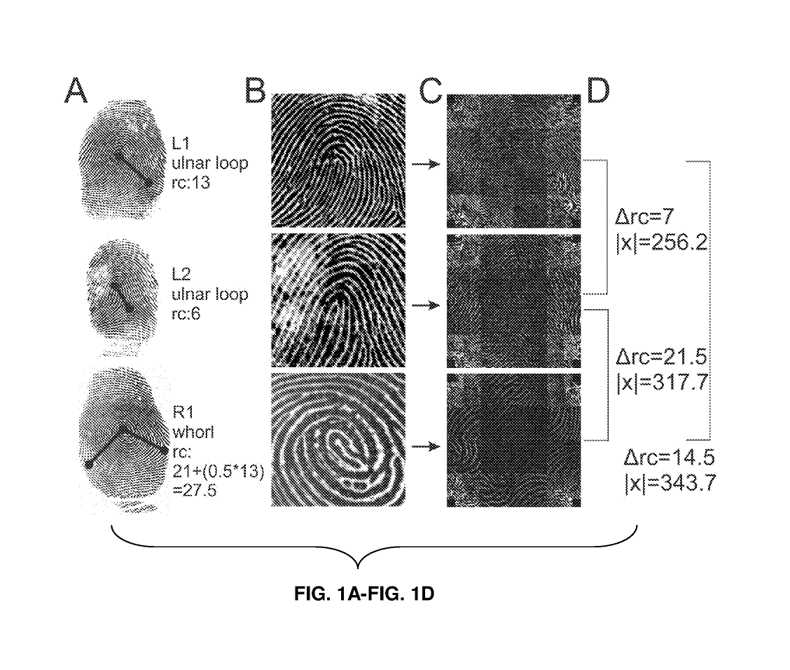

Pattern analysis can only compare the fingerprint pattern (simplest classification includes arch, loop or whorl) or generates ridge counts that are then compared between prints, as shown in FIGS. 1A-1D). It is to be noted that there are severe limitations to using only pattern analysis, as pattern analysis is a very coarse measurement (using only about 30 different values), and depending on the fingerprint image, the count can be fairly subjective and easily vary by ±2.

In contrast, in the present detection system, a wavelet-based analysis is used which measures different features; yet still provides a less complex description of the fingerprint (e.g., a feature vector of 36 numbers, shown in FIG. 1A-FIG. 1D) which can then be compared with a second print by calculating the Euclidean or Manhattan distance. (It is to be understood that other methods to determine the distance between two vectors are also within the contemplated scope of the present invention). The wavelet-based method has several advantages: ability to detect overall differences, is mostly immune to variation in acquisition (i.e., finger placement on the scanner), and provides a similarity score that can be used as a score of symmetry.

In one embodiment, to assess FA in fingerprints as a risk score for T2DM, receiver operating characteristic (ROC) curves can be created for each finger, in which the true-positive rate and the false-positive rate are paired across all potential cutoff points that distinguished between individuals with and without T2DM. In the ROC curve, the true-positive rate (sensitivity) is the proportion of individuals with T2DM that the FA correctly predicted as having T2DM; the higher the rate, the more accurate the test. The false-positive is the proportion of the control individuals that the FA score incorrectly predicted as have T2DM; the lower the rate, the more accurate the test. The true-negative rate (specificity) is therefore the proportion of the controls that FA correctly classified as not having T2DM. It is desired that the detection system have a high true-positive rate and a low false-positive rate in partitioning individuals with, and without, T2DM.

Test for T2DM Susceptibility

In addition, the detection system described herein can be useful to determine when during gestation embryos are most susceptible to environmental stressors (e.g., mother with diabetes) that interact with the genes for diabetes, indicating when during gestation therapeutic intervention aimed at decreasing the incidence of diabetes must be instituted. It is now believed that asymmetry in bilateral traits can be a strong indicator of a genotype that optimizes growth over development. In addition, while not wishing to be bound by theory, it may be that plasticity is superior to discrete alternative reproductive tactics, and demonstrates the importance of identifying alternative growth strategies within a species.

Fingerprint Images

In certain embodiments, to obtain a print showing all friction ridges of an individual finger, a rolled fingerprint image can be used. While this rolled fingerprint image is more time consuming than acquiring slap fingerprint images, use of a rolled fingerprint image can ensure a precise classification of the ridge pattern and an accurate ridge count. For example, the fingerprint images of all fingers on both hands can be stored as uncompressed digital images on a laptop dedicated to the project with an encrypted storage device.

Fingerprints were collected using the Crossmatch Verifier 320 scanner. The prints of fingers on both hands were stored as uncompressed digital images on a laptop dedicated to the project with an encrypted storage device. Fingerprints were scored for similarity between homologous fingers (symmetry) using both ridge counts (pattern analysis) and wavelet based methods.

Data Analysis

The prints from 101 individuals from this cohort (out of 240 collected) have been scored for differences in ridge counts and Haar wavelet decomposition (similarity scores). The results show that asymmetry in fingerprints is an indicator of propensity to develop diabetes. Described herein are the analyses of the wavelet decomposition scoring, as compared to pattern analysis.

First, to consider the predictability of asymmetry in fingerprints for developing diabetes use multivariable logistic regressions can be used. In the model for finger 1 (thumb), the significant independent predictors of diabetes state (T2DM or control) were age (Wald X²=24.95, P=0.0001) and asymmetry score (Wald X²=5.62, P=0.018). Sex was not significant (P=0.545). This analysis shows that an increase in one point for the asymmetry score for finger 1 (cohort mean=227.7, SE=83.2) increases the odds of being T2DM by a multiplicative factor of 1.0 (Exp (B)=1.012).

Second, to consider the predictability of asymmetry in fingerprints to provide additional information about environmental changes during gestation, certain variables (e.g. age, sex, and the like) were measured. In particular, variations in symmetry scores for each set of homologous fingerprint images were determined using multimodal regression analysis.

Additional Data Analysis

Fingerprints do not change throughout an individual's lifetime; therefore the detection system described herein is also useful for predicting risk of developing diabetes at any postnatal age and prior to development of any phenotypic correlates with diabetes.

Also, in certain embodiments, one or more additional variables can be co-determined and used with the detection system described herein. As such, the control data can specifically include older individuals without diabetes and/or older individuals without diabetes that have healthy lifestyles. It is to be noted that it is more difficult to obtain readable fingerprints from older adults. Therefore, in certain embodiments, scanners with higher resolution can be useful in order to obtain the fingerprints.

Data Analysis

Receiver operating characteristic (ROC) curves for each finger are created, in which the true-positive rate (sensitivity) and the false-positive rate (1— specificity) are paired across all potential cutoff points that distinguished between individuals with and without T2DM. Given that age was significant in the multinomial regression, only individuals over 40 years of age were included in this analysis.

In certain embodiments, the detection system can be used in concert with genomic sequencing that has identified genes associated with T2DM. While not wishing to be bound by theory, it is now believed that genomic sequencing will have more false positives than the fingerprint detection system described herein because possessing the genes for T2DM may be necessary, but not sufficient for, the development of diabetes. Thus, a comparison between the two methods not only supports fingerprint asymmetry as a powerful new detection system for detecting risk, but also shows that gene x environment interactions (which fingerprints can detect) are important when explaining variation in the propensity to develop T2DM across humans.

### EXAMPLES

Certain embodiments of the present invention are defined in the Examples herein. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

Materials and Methods

Individuals were seen either at the UMA Diabetes/Endocrine Center or the UMA Family Medicine Center in Athens Ohio. Individuals were included in the control group if they had no individual or family history of diabetes or any insulin resistant syndrome (such as polycystic ovarian syndrome, metabolic syndrome). This was confirmed clinically by a clinical interview/exam by the Diabetes Endocrine Center faculty or a detailed chart review. The individual's medical records were used to exclude those individuals with chromosomal syndromes (e.g., Down, Turner, Klinefelter), monogenic disease (e.g., cystic fibrosis, MODY), polygenic morbidity (e.g., cleft palate and cleft lip with or without cleft palate), and females suffering from endometrial carcinoma or carcinoma of cervix, all of which are correlated with fingerprint asymmetries. Fingerprints were collected using the Crossmatch Verifier 320 scanner. The fingerprints of fingers on both hands were stored as uncompressed digital images on a laptop dedicated to the project with an encrypted storage device.

Fingerprints were scored for similarity between homologous fingers (symmetry) using both ridge counts (pattern analysis) and wavelet based methods.

Gestational Development and T2DM

Fingerprints are influenced by an interaction between genetics and the inter-uterine environment. Once formed, however, fingerprints do not change over an individual's lifetime.

There is uncertainty about precisely when during gestation the environment impacts the metabolism of the adult. As there are preventive measures that could be taken during pregnancy, fingerprints could help to more clearly identify the timing of this critical period during gestation.

To determine that some stages of gestational development are more important in the onset of T2DM, the degree of FA of the different fingers can be compared.

Thus, in certain embodiments, such detection system is useful to identify the gestational stages when the uterine environment is more likely to influence an individual's propensity to develop diabetes. While not wishing to be bound by theory, it is now believed that the ridge pattern of fingerprints results from stress and tension lines in volar pads. These volar pads are transitional swellings of the embryo's hand mesenchyme that begin 7-8 weeks into gestation. Interestingly, volar pad development begins at the thumb and progresses towards the little finger, allowing developmental deficiencies to arise during different times of gestation to present as asymmetry for different fingers. Volar pad size is determined by many factors, among them diet of the mother and other factors that affect growth rate. This suggests volar pads are influenced by the same environmental factors that influence diabetes. At 10-10.5 weeks estimated gestational age (EGA) primary ridges form through rapid division of epidermal cells. This is possibly correlated with or even triggered by innervation from spinal cord levels C6, C7, and C8. Each of these spinal cord levels innervates different fingers. By 16 weeks EGA, the tissue surrounding the volar pads has caught up in growth and the originally enlarged volar pads now blend in with the finger contours. The timing (determined by growth rate) of this process influences the ridge pattern. It is now believed that volar pad height, size, and shape determine the friction ridge shape by influencing the stress across the skin. High volar pads result in high ridge counts and whorl patterns, low pads to low counts and arch patterns.

In addition, environmental stress during gestation can influence fingerprint ridge counts. Environmental influences during gestation are related to risk for T2DM as well, and can affect subsequent adult metabolic rates as well as adult body size. A baby of reduced birth size, for example, carries an increased risk of insulin resistance in later life. Still, there is uncertainty about precisely when during gestation the environment impacts the metabolism of the adult. As there are preventive measures that could be taken during pregnancy, more clearly identifying the timing of this critical period during gestation can be very important.

By comparing different aspects of fingerprints ridge count and shape across fingers, it is now possible to determine when during gestation the environment was unfavorable, leading to diabetics for those with specific genes.

In certain embodiments, the method further comprises wherein the fingerprint images are obtained from the fetus over time.

Some stages of gestational development appear to be more important in the onset of T2DM. When the degree of FA of the different fingers were compared to determine which finger was the most predictive of T2DM, finger pairs 4 and 4 were predictive; for T1DM, finger pairs 3, 4 and 5 were predictive.

Data Acquisition and Processing

Rolled fingerprints were collected using a Crossmatch Verifier 320 LC scanner at a resolution of 500 ppi and 256 level grayscale. The prints of all fingers on both hands were stored as uncompressed digital images (bmp file format) on a laptop dedicated to the project with an encrypted storage device. Fingerprints were scored for similarity between corresponding fingers on each hand (symmetry) using both ridge counts (pattern analysis) and a wavelet based method. This method generates ridge counts that are then compared between prints (See FIG. 1). The software package RIDGECOUNTER® was used to score finger tips with a loop pattern the ridge count equal to the number of ridges crossing the single straight line between the core and the triradius, and for fingertip patterns with two triradial points (whorl and double loop pattern), ridge counts equaled the counts crossing both the lines. The absolute difference between the scores for the two hands was used as the asymmetry score. As fingertips with an arch are standardly assigned a ridge count of zero, finger pairs with two arches would have produced an “artificial” score of symmetry, and with one arch, and inflated score of asymmetry. Therefore, arch fingerprints were not included in the pattern-based data.

For wavelet analysis, a 256 pixel square sub-image derived from the center of each print to calculate a feature vector (based on Haar wavelets) was processed using MATLAB and the Wavelet Toolbox extension.

Statistical Analyses

An age relationship is expected for T2DM, as the risk for developing T2DM increases with age. In addition, females are less likely to develop diabetes at the same BMI that males develop diabetes Therefore, both age and gender were controlled for in the analyses. Multinomial logistic regressions were conducted for each finger pair to determine if asymmetry scores could be considered a risk factor for diabetes, and to determine which fingers were the most predictive (highest coefficient). The parameters that were entered into all models, in addition to the asymmetry score for a finger pair, included age, gender and an interaction between age and asymmetry scores. The discriminative accuracy of the asymmetry scores for each finger pair was also assessed using the area under the receiver operating characteristic curves (AUCs).

The asymmetry scores were compared for each finger pair between individuals with T2DM and T1DM with independent t-tests. The pattern asymmetry scores were normalized with the transformation (LN (score+0.33)) and wavelet asymmetry scores with the transformation (LN(score)).

Results

A total of 340 individuals were finger printed, 83 were classified as controls (Average Age=31.3 years+/−16.8), 200 had been diagnosed with T2DM (Average Age=59.2 years+/−13.59) and 57 diagnosed with T1DM (Average Age=42.4+/−16.93). In this sample there were 133 males (26=controls; 78=T2; 29=T1) and 207 females (57=controls; 122=T2; 28=T1).

Ridge Counts (Patterns) to Determine Asymmetry

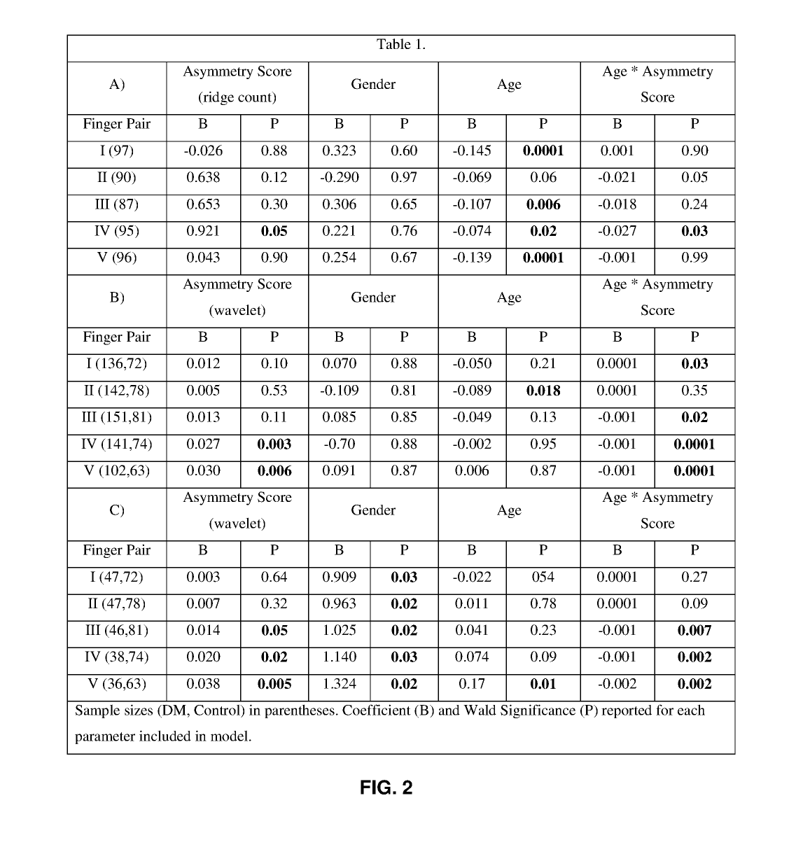

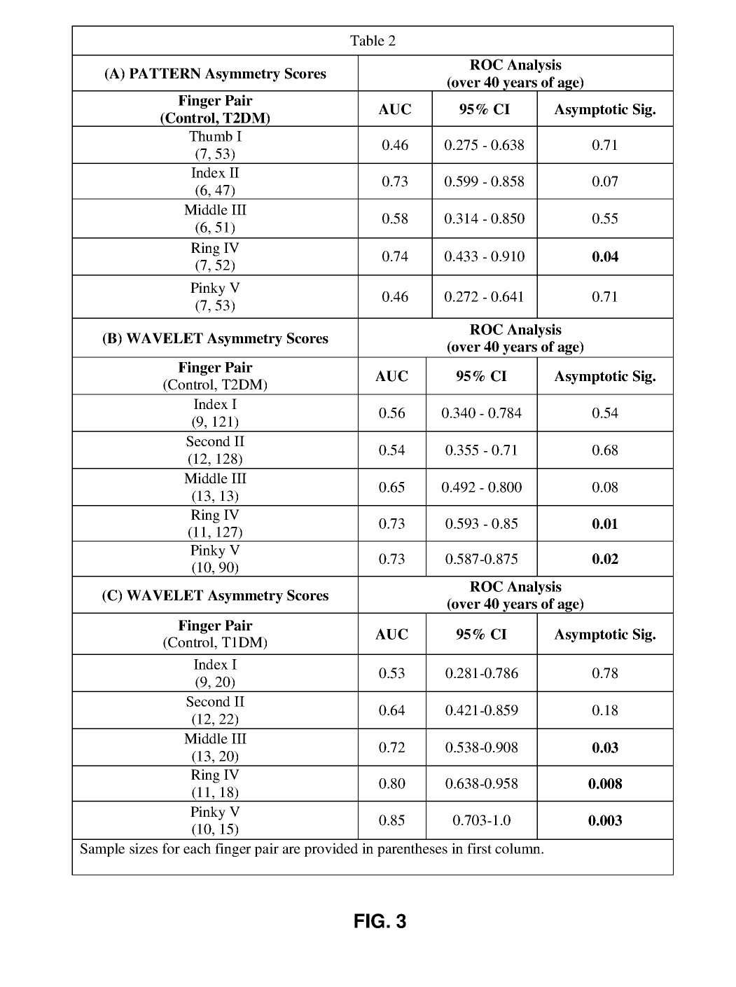

Individuals that did not have a complete set of prints (all ten fingers) that could be scored for ridge counts, due to either poor quality prints or an arch type print, were removed from the pattern analysis. Ridge count data were collected for 85 females and 51 males, and 44 of these individuals were classified as “Controls”, 62 with T2DM, and 21 with T1DM. In the multinomial regression analyses of each pair of fingers, asymmetry scores were only significant in the model for finger pair IV (FIG. 2—Table 1A). Gender was not significant for any of the finger pairs, however a significant influence of age was detected for all finger pairs except II, and an interaction between age and asymmetry score for finger pair IV (FIG. 2—Table 1A). These results show that some of the younger controls may not be “true” controls (i.e., individuals with a high risk of developing diabetes, but that have not yet developed the disease). Therefore, to remove the potential bias of “false” controls, the data set was split into two groups (>40 years of age and <40 years of age) and conducted the receiver operating characteristic curves (ROC) on the subset of individuals over 40 years of age. The AUC score for the subset of individuals over 40 for finger IV was the most predictive, and was significantly better than the null ((FIG. 3—Table 2A).

Wavelet Analysis of Asymmetry

More of the prints could be analyzed using wavelet analysis than the ridge count analysis, as the wavelet method is less reliant on getting a clear print or having a particular fingerprint pattern. In the multinomial regression analyses, in which the ability of asymmetry scores for each pair of fingers were assessed to predict T2DM diabetes state, the asymmetry scores significantly predicted T2DM for finger IV and V ((FIG. 2—Table 1B). Gender was not significant for any of the fingers, however an interaction between age and asymmetry score was significant for the models of all but one of the finger pairs ((FIG. 2—Table 1B). Therefore, to remove potentially “false” controls (younger individuals that have a high risk of diabetes but have not yet developed diabetes), only individuals over the age of 40 years were analyzed in the ROC analyses. Area under the curves ranged from 0.54 to 0.73, and the AUC for finger IV and V were significantly greater than the null (0.5) ((FIG. 3—Table 2B).

Asymmetry scores were significant in predicting T1DM diabetes for finger pairs III, IV and V in the multinomial regression analyses ((FIG. 2—Table 1C). Gender was significant for every finger pair; a higher proportion of males with T1DM than females was detected in the data. In addition there were significant interactions between age and asymmetry scores for the models for all of the finger pairs except finger II ((FIG. 2—Table 1C). Therefore, the ROC curves for the subset of individuals over 40 years of age were examined. Area under the curves ranged from 0.53 to 0.85, and the AUC scores for finger pairs III, IV and V were significantly greater than the null (0.5) (FIG. 3—Table 2C).

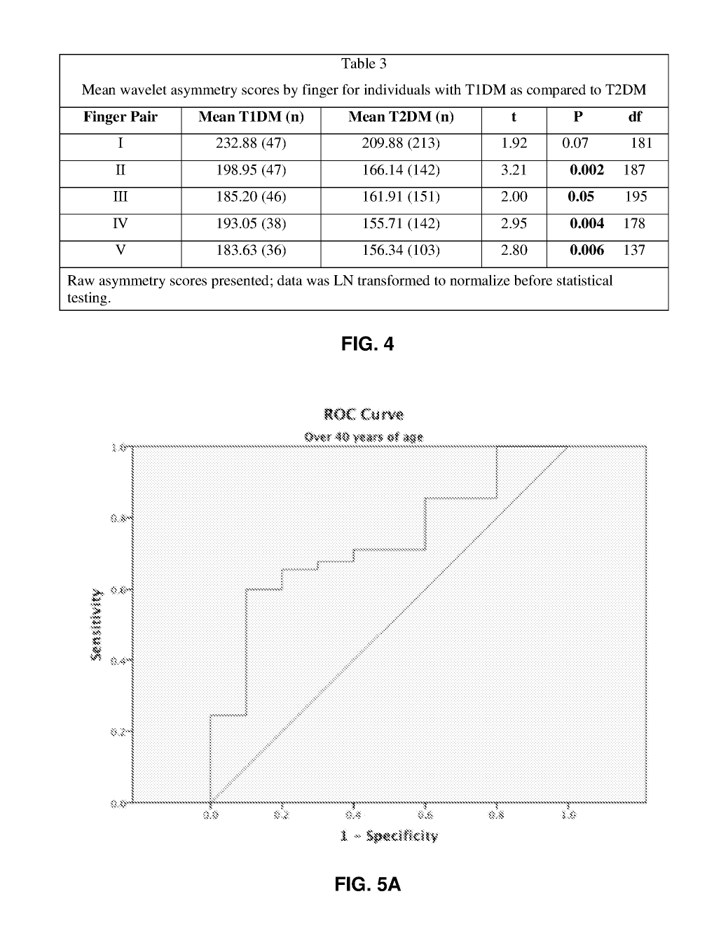

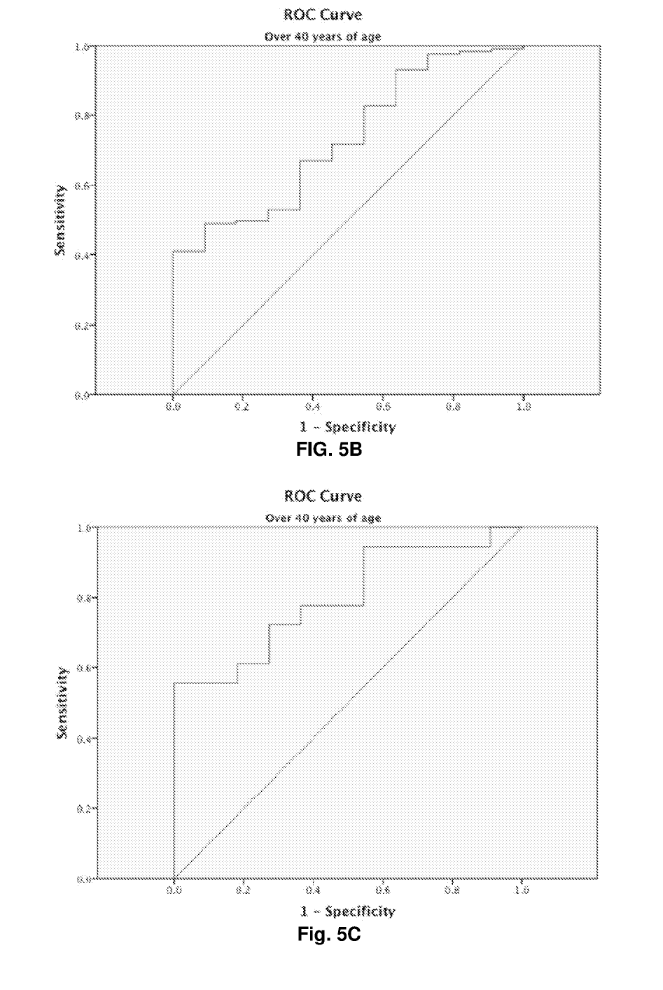

T1DM individuals were statistically more asymmetrical than T2DM individuals for every finger pair except finger I (FIG. 4—Table 3).

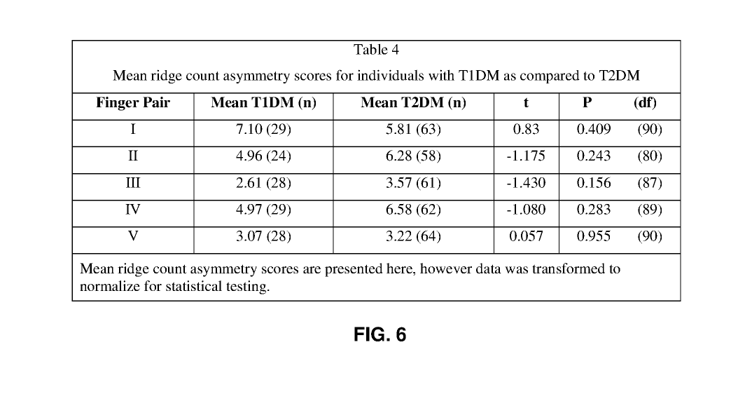

There were no significant difference in the pattern asymmetry scores between T2DM and T1DM for any of the finger pairs (FIG. 6—Table 4).

### Discussion

Assessing asymmetry of fingerprint scores is useful as a valuable diagnostic tool for health providers in their assessment of an individual's risk of developing diabetes. Asymmetry scores calculated using both a wavelet-based method and the standard ridge count method, shows that finger IV is the most predictive of T2DM in this population.

Risk prediction based on fingerprints can predict from birth and are fixed for life. Also, finally, compared to genetic testing, that can also predict as early at birth, AUC scores for the fingerprint asymmetry of finger pair IV that were detected are slightly higher than for genetic polymorphisms alone (AUC=0.60, 95% CI=0.57-0.63³³) and would have substantially lower costs.

Asymmetry scores for finger IV were the most predictive of T2DM, showing that the time period during gestation when the prints on these fingers are forming may be a critical time stage of development for increasing risk of developing diabetes. Fingerprints begin to form between 6 and 7 weeks, and are complete by 16-17 weeks, beginning with the thumb (finger I) and progressing to the little finger (finger V). These results show that the critical time is towards the end of this time period (14-16 weeks). There were differences between the finger pair were detected as most predictive.

Described herein is the ability of the asymmetry scores to predict risk of T1DM, and the overall higher asymmetry for the prints of individuals with T1DM as compared to individuals with T2DM. While T1DM and T2DM are considered quite different diseases, they are both multifactoral diseases with a complex interaction between predisposing genetic and environmental factors. In addition, an increased frequency of T2DM diabetes has been detected in families with T1DM diabetes, which could suggest a common genetic susceptibility. Finally, a large proportion of diabetic patients may have both T1DM and T2DM processes contributing to their diabetic phenotype, and therefore individuals that were diagnosed as T1DM in these examples may have the propensity to develop T2DM later in life.

Thus, fingerprint asymmetry is a valuable diagnostic tool for predicting risk of T2DM and T1DM, and that wavelet analysis is a method that is useful to assess asymmetry in fingerprints.

One advantage of fingerprints scored using wavelet-based methods over genetic testing is that this test is much less expensive than using genetic testing. This is important, given recent reports that both risk-aware and risk-unaware individuals were interested in genetic testing, but identified the need for low-cost tests.

Diagnostic Applications

A diagnostic tool based on FA in the fingerprints of finger pair IV, measured using a wavelet analysis is useful for predicting risk prior to associated health problems for both T2DM and T1DM.

In addition, given that that the prints form for fetal fingers IV and V develop during the 14-17 weeks of gestation, interventions during this time period of pregnancy will be most successful.

Electronic Apparatus Readable Media, Systems, Arrays and Methods of Using the Same

A “computer readable medium” is an information storage media that can be accessed by a computer using an available or custom interface. Examples include memory (e.g., ROM or RAM, flash memory, etc.), optical storage media (e.g., CD-ROM), magnetic storage media (computer hard drives, floppy disks, etc.), punch cards, and many others that are commercially available. Information can be transmitted between a system of interest and the computer, or to or from the computer to or from the computer readable medium for storage or access of stored information. This transmission can be an electrical transmission, or can be made by other available methods, such as an IR link, a wireless connection, or the like.

“System instructions” are instruction sets that can be partially or fully executed by the system. Typically, the instruction sets are present as system software.

The system can also include detection apparatus that is used to detect the desired information, using any of the approaches noted herein. For example, a detector configured to obtain and store fingerprint images or a fingerprint reader can be incorporated into the system. Optionally, an operable linkage between the detector and a computer that comprises the system instructions noted above is provided, allowing for automatic input of specific information to the computer, which can, e.g., store the database information and/or execute the system instructions to compare the detected specific information to the look up table.

Optionally, system components for interfacing with a user are provided. For example, the systems can include a user viewable display for viewing an output of computer-implemented system instructions, user input devices (e.g., keyboards or pointing devices such as a mouse) for inputting user commands and activating the system, etc. Typically, the system of interest includes a computer, wherein the various computer-implemented system instructions are embodied in computer software, e.g., stored on computer readable media.

In certain embodiments, the computer readable medium includes at least a second reference profile that represents a level of at least one additional fingerprint asymmetry score from one or more samples from one or more individuals exhibiting indicia of DM.

In another aspect, there is provided herein a computer system for determining whether an individual is predisposed to having DM, comprising a database and a server comprising a computer-executable code for causing the computer to receive a profile of an individual, identify from the database a matching reference profile that is diagnostically relevant to the individual profile, and generate an indication of whether the individual is predisposed to having DM.

In another aspect, there is provided herein a computer-assisted method for evaluating the presence, absence, nature or extent of DM in an individual, comprising: i) providing a computer comprising a model or algorithm for classifying data from a sample obtained from the individual, wherein the classification includes analyzing the data for the presence, absence or amount of at least asymmetry homologous fingerprint score; ii) inputting data from the fingerprint image sample obtained from the individual; and, iii) classifying the biological sample to indicate the presence, absence, nature or extent of DM.

As used herein, “electronic apparatus readable media” refers to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; and general hard disks and hybrids of these categories such as magnetic/optical storage media. The medium is adapted or configured for having recorded thereon a marker as described herein.

As used herein, the term “electronic apparatus” is intended to include any suitable computing or processing apparatus or other device configured or adapted for storing data or information. Examples of electronic apparatus suitable for use with embodiments of the present invention include stand-alone computing apparatus; networks, including a local area network (LAN), a wide area network (WAN) Internet, Intranet, and Extranet; electronic appliances such as personal digital assistants (PDAs), cellular phone, pager and the like; and local and distributed processing systems.

As used herein, “recorded” refers to a process for storing or encoding information on the electronic apparatus readable medium. Those skilled in the art can readily adopt any method for recording information on media to generate materials comprising the markers described herein.

A variety of software programs and formats can be used to store the fingerprint image information on the electronic apparatus readable medium. Any number of data processor structuring formats (e.g., text file or database) may be employed in order to obtain or create a medium having recorded thereon the markers. By providing the markers in readable form, one can routinely access the information for a variety of purposes. For example, one skilled in the art can use the information in readable form to compare a sample fingerprint image with the control information stored within the data storage means.

Thus, there is also provided herein a medium for holding instructions for performing a method for determining whether an individual has a pre-disposition for developing DM, wherein the method comprises the steps of determining the presence or absence of asymmetry in homologous fingerprints, and based on the presence or absence of such asymmetry, determining whether the individual has a pre-disposition for developing DM, and/or recommending a particular treatment for DM or pre-DM condition. It is contemplated that different entities may perform steps of the contemplated methods and that one or more means for electronic communication may be employed to store and transmit the data. It is contemplated that raw data, processed data, diagnosis, and/or prognosis would be communicated between entities which may include one or more of: a primary care physician, patient, specialist, insurance provider, foundation, hospital, database, counselor, therapist, pharmacist, and government.

There is also provided herein an electronic system and/or in a network, a method for determining whether an individual has a pre-disposition for developing DM wherein the method comprises the steps of determining the presence or absence of asymmetry in homologous fingerprints, and based on the presence or absence of such asymmetry, determining whether the individual has a pre-disposition for developing DM, and/or recommending a particular treatment for DM or pre-DM condition. The method may further comprise the step of receiving information associated with the individual and/or acquiring from a network such information associated with the individual.

Also provided herein is a network, a method for determining whether an individual has a pre-disposition for developing DM associated with asymmetry in one or more homologous fingerprints, the method comprising the steps of receiving information associated with the homologous fingerprints, receiving phenotypic information associated with the individual, acquiring information from the network corresponding to the homologous fingerprints and/or DM, and based on one or more of the phenotypic information, the homologous fingerprints, and the acquired information, determining whether the individual has a pre-disposition for developing DM. The method may further comprise the step of recommending a particular treatment for the DM or pre-DM disease condition.

There is also provided herein a business method for determining whether an individual has a pre-disposition for developing DM, the method comprising the steps of receiving information associated with fingerprint images of homologous fingerprints, receiving phenotypic information associated with the individual, acquiring information from the network corresponding to the homologous fingerprints and/or DM, and based on one or more of the phenotypic information, the homologous fingerprints, and the acquired information, determining whether the individual has a pre-disposition for developing DM. The method may further comprise the step of recommending a particular treatment for DM or pre-DM condition.

Kits

Particular embodiments are directed to kits useful for the practice of one or more of the methods described herein. Kits for using detection method described herein for therapeutic, prognostic, or diagnostic applications and such uses are contemplated by the inventors herein. The kits can include devices for capturing fingerprint images, as well as information regarding a standard or normalized profile or control.

Also, the kits can generally comprise, in suitable means, distinct containers or image collecting devices for each individual fingerprint image. The kit can also include instructions for employing the kit components as well the use of any other reagent not included in the kit. Instructions may include variations that can be implemented. It is contemplated that such reagents are embodiments of kits of the invention. Also, the kits are not limited to the particular items identified above and may include any reagent used for the manipulation or characterization of the fingerprint images and/or data derived therefrom.

The kits described herein can reduce the costs and time associated collecting a variety of images. The kits may be used by research and commercial laboratories and medical end users to facilitate collection of fingerprint data in remote locations.

The methods and kits of the current teachings have been described broadly and generically herein. Each of the narrower species and sub-generic groupings falling within the generic disclosure also form part of the current teachings. This includes the generic description of the current teachings with a proviso or negative limitation removing any subject matter from the genus, regardless of whether or not the excised material is specifically recited herein.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

What is claimed is:

1. A method for determining a propensity to develop diabetes mellitus (DM) in an individual, comprising:
a) measuring an asymmetry between one or more captured fingerprint images from homologous fingers of an individual, wherein the measuring comprises using wavelet analysis to determine a degree of a fluctuating asymmetry;
b) calculating an amount of asymmetry measured in step (a) relative to the amount of asymmetry in a control sample, wherein the calculating comprises setting a boundary value between: i) a degree of asymmetry in homologous fingerprint images collected from a control population; and ii) a degree of asymmetry in the homologous fingerprint image collected from the individual; and,
c) determining the propensity for developing DM where the degree of asymmetry of the homologous fingerprint image/s measured is high as compared to the fingerprint image/s collected from the control population.

2. The method of claim 1, wherein the asymmetry is measured any time after birth of the individual.

3. The method of claim 1 or 2, further comprising comparing the first homologous fingerprint image with the second homologous fingerprint image by calculating the Euclidean or Manhattan distances between the first homologous fingerprint image and the second homologous fingerprint image.

4. The method of claim 1, 2 or 3, wherein the homologous fingerprints are of the fourth finger.

5. The method of claim 4, wherein the homologous fingerprints are of the fourth and fifth fingers, and the DM is Type 2 diabetes mellitus.

6. The method of claim 4, wherein the homologous fingerprints are of the third, fourth and/or fifth fingers, and the DM is Type 1 diabetes mellitus.

7. The method of claim 1, further comprising:
receiving phenotypic information associated with the individual, and based on one or more of the phenotypic information, the homologous fingerprints, and the acquired information, and
determining whether the individual has a pre-disposition for developing DM, and, optionally,
recommending a particular treatment for the DM or pre-DM disease condition.

8. The method of claim 1, further including indicating when a therapeutic intervention aimed at decreasing the incidence of diabetes is beneficial.

9. A kit for determining the whether an individual is at risk for developing diabetes mellitus (DM), comprising:
an image capturing device for: i) obtaining at least a first image of at least one fingerprint of at least one finger of the individual; and, i) obtaining at least a second image of at least one fingerprint of at least one homologous finger of the individual;
a device for comparing the at least first fingerprint image to the at least second image, wherein the comparing comprises using wavelet analysis; and,
instructions for use of the wavelet analysis in determining the risk of developing DM.

10. The kit of claim 9, wherein the instructions include determining fluctuating asymmetry in the fingerprint images.

11. The kit of claim 9 or 10, wherein the fingerprint images are laid down in a database.

12. A system for evaluating a patient at risk of developing diabetes mellitus, the system comprising:
a data storage device storing instructions for evaluating a patient at risk of developing diabetes mellitus; and
a processor configured to execute the instructions to perform a method including:
a) receiving patient-specific data regarding images of the patient's fingerprints;
b) creating a stored image representing at least a portion of the fingerprint based on the received patient-specific data; and
c) classifying the risk of developing diabetes mellitus based on the asymmetry in the patient's fingerprint images by conducting a wavelet analysis of characteristic within the patient's fingerprints;
and, optionally,
d) generating a treatment recommendation or risk assessment based on the wavelet analysis value of the fingerprint's asymmetry and the classification of the diabetes mellitus.

13. The system of claim 12, wherein the classifying comprises:
measuring an asymmetry between one or more captured fingerprint images from homologous fingers of an individual, wherein the measuring comprises using wavelet analysis to determine a degree of a fluctuating asymmetry; and,
calculating an amount of asymmetry relative to the amount of asymmetry in a control sample, wherein the calculating comprises setting a boundary value between: i) a degree of asymmetry in homologous fingerprint images collected from a control population; and ii) a degree of asymmetry in the homologous fingerprint image collected from the individual.

14. The system of claim 12 or 13, wherein the asymmetry is measured any time after birth of the individual.

15. The system of claim 12, 13 or 14, further comprising comparing the first homologous fingerprint image with the second homologous fingerprint image by calculating the Euclidean or Manhattan distances between the first homologous fingerprint image and the second homologous fingerprint image.

16. The system of claim 12, 13, 14 or 15, wherein the homologous fingerprints are of the fourth finger.

17. The system of claim 16, wherein the homologous fingerprints are of the fourth and fifth fingers, and the DM is Type 2 diabetes mellitus.

18. The system of claim 16, wherein the homologous fingerprints are of the third, fourth and/or fifth fingers, and the DM is Type 1 diabetes mellitus.

19. A computer-implemented method of determining a risk of developing diabetes mellitus in an individual, the method comprising:
a) receiving patient-specific data regarding images of the patient's fingerprints;
b) creating a stored image representing at least a portion of the fingerprint based on the received patient-specific data; and
c) classifying the risk of developing diabetes mellitus based on the asymmetry in the patient's fingerprint images by conducting a wavelet analysis of characteristics within the patient's fingerprints;
and, optionally,
d) generating a treatment recommendation or risk assessment based on the wavelet analysis value of the fingerprint's asymmetry and the classification of the diabetes mellitus.

20. The computer-implemented method of claim 19, wherein the treatment recommendation includes medication, dietary changes, or an exercise regimen.

21. The computer-implemented method of claim 19 or 20, wherein the classification of the disease includes determining Type 1 diabetes mellitus or Type 2 diabetes mellitus.

22. The computer-implemented method of claim 19, 20 or 21, wherein the classifying comprises:
measuring an asymmetry between one or more captured fingerprint images from homologous fingers of an individual, wherein the measuring comprises using wavelet analysis to determine a degree of a fluctuating asymmetry; and,
calculating an amount of asymmetry relative to the amount of asymmetry in a control sample, wherein the calculating comprises setting a boundary value between: i) a degree of asymmetry in homologous fingerprint images collected from a control population; and ii) a degree of asymmetry in the homologous fingerprint image collected from the individual.

23. The computer-implemented method of claim 19, 20, 21 or 22, wherein the asymmetry is measured any time after birth of the individual.

24. The computer-implemented method of claim 19, 20, 21, 22 or 23, further comprising comparing the first homologous fingerprint image with the second homologous fingerprint image by calculating the Euclidean or Manhattan distances between the first homologous fingerprint image and the second homologous fingerprint image.

25. The computer-implemented method of claim 19, 20, 21, 22, 23 or 24, wherein the homologous fingerprints are of the fourth finger.

26. The computer-implemented method of claim 25, wherein the homologous fingerprints are of the fourth and fifth fingers, and the DM is Type 2 diabetes mellitus.

27. The computer-implemented method of claim 25, wherein the homologous fingerprints are of the third, fourth and/or fifth fingers, and the DM is Type 1 diabetes mellitus.

28. A non-transitory computer readable medium for use on a computer system containing computer-executable programming instructions for performing a method of determining a risk of developing diabetes mellitus in an individual, the method comprising:
a) receiving patient-specific data regarding images of the patient's fingerprints;
b) creating a stored image representing at least a portion of the fingerprint based on the received patient-specific data; and
c) classifying the risk of developing diabetes mellitus based on the asymmetry in the patient's fingerprint images by conducting a wavelet analysis of characteristic within the patient's fingerprints;
and, optionally
d) generating a treatment recommendation or risk assessment based on the wavelet analysis value of the fingerprint's asymmetry and the classification of the diabetes mellitus.

29. The non-transitory computer readable medium of claim 28, wherein the treatment recommendation includes medication, dietary changes, or an exercise regimen.

30. The non-transitory computer readable medium of claim 28 or 29, wherein the classification of the disease includes determining Type 1 diabetes mellitus or Type 2 diabetes mellitus.

31. The non-transitory computer readable medium of claim 28, 29 or 30, wherein the classifying comprises:
measuring an asymmetry between one or more captured fingerprint images from homologous fingers of an individual, wherein the measuring comprises using wavelet analysis to determine a degree of a fluctuating asymmetry; and,
calculating an amount of asymmetry relative to the amount of asymmetry in a control sample, wherein the calculating comprises setting a boundary value between: i) a degree of asymmetry in homologous fingerprint images collected from a control population; and ii) a degree of asymmetry in the homologous fingerprint image collected from the individual.

32. The non-transitory computer readable medium of claim 28, 29, 30 or 31, wherein the asymmetry is measured any time after birth of the individual.

33. The non-transitory computer readable medium of claim 28, 29, 30, 31 or 32, further comprising comparing the first homologous fingerprint image with the second homologous fingerprint image by calculating the Euclidean or Manhattan distances between the first homologous fingerprint image and the second homologous fingerprint image.

34. The non-transitory computer readable medium of claim 28, 29, 30, 31, 32, or 33, wherein the homologous fingerprints are of the fourth finger.

35. The non-transitory computer readable medium of claim 34, wherein the homologous fingerprints are of the fourth and fifth fingers, and the DM is Type 2 diabetes mellitus.

36. The non-transitory computer readable medium of claim 34, wherein the homologous fingerprints are of the third, fourth and/or fifth fingers, and the DM is Type 1 diabetes mellitus.

37. An electronic system for use in determining whether an individual has a pre-disposition for developing DM, comprising:
an image capturing device for determining the presence or absence of asymmetry in homologous fingerprints, and based on the presence or absence of such asymmetry, and
a computer implemented system determining whether the individual has a pre-disposition for developing DM, and, optionally, recommending a particular treatment for DM or pre-DM condition.
